SEQUENCE LISTING (25 sequences)

SEQ ID NO: 1 - protein, 97 aa, Homo sapiens
PRAPGNLTVHTNVSDTLLLTWSNPYPPDNYLYNHLTYAVNIWSENDPADFRIYNVTYLEPSLRIAASTLKSGISYRARVRAWAQCYNTTWSEWSPST

SEQ ID NO: 2 - protein, 175 aa, Homo sapiens
SNWGCYGNIQSLDTPGASCGIGRRHGLNYCGVRASERLAEIDMPYLLKYQPMMQTIGQKYCMDPAVIAGVLSRKSPGDKILVNMGDRTSMVQDPGSQAPTSWISESQVSQTTEVLTTRIKEIQRRFPTWTPDQYLRGGLCAYSGGAGYVRSSQDLSCDFCNDVLARAKYLKRHGF

SEQ ID NO: 3 - protein, 237 aa, Homo sapiens
MFVDNRIQKSMLLDLNKEIMNELGVTVVGDIIAILKHAKVVHRQDMCKAATESVPCSPSPLAGEIRRGTSAASRMITNSLNHDSPPSTPPRRPDTSTSKISVTVSNKMAAKSAKATAALARREEESLAVPAKRRRVTAEMEGKYVINMPKGTTPRTRKILEQQQAAKGLHRTSVFDRLGAETKADTTTGSKPTGVFSRLGATPETDEDLAWDSDNDSSSSVLQYAGVLKKLGRGPAK

SEQ ID NO: 4 - protein, 79 aa, Homo sapiens
SQGVCSKQTLVVPLHYNESYSQPVYKPYLTLCAGRRICSTYRTMYRVMWREVRREVQQTHAVCCQGWKKRHPGALTCEA

SEQ ID NO: 5 - protein, 76 aa, Homo sapiens
HEAHKTSLSSWKHDQDWANVSNMTFSNGKLRVKGIYYRNADICSRHRVTSAGLTLQDLQLWCNLRSVARGQIPSTL

SEQ ID NO: 6 - protein, 101 aa, Homo sapiens
GVAEFHMSLTVSCPDPTPSTDPQGRHNREPILGRDDDFMCKQVKFRMCVVGRDGNAQSSVRYTGPLYRRKIRTEFLFVVFLLETRELKPQVNKNVQGTRPS

SEQ ID NO: 7 - protein, 92 aa, Homo sapiens [sequence omitted]

SEQ ID NO: 8 - protein, 92 aa, Homo sapiens
ATGILICMTKNLESVHSIVLAHSCYHHENKPRPDCCFQQKIRDTKSKVELPRHAHARLTNPQLTHRSMKINDCCIKPLRFGVTCYAAFCDNN

SEQ ID NO: 9 - protein, 237 aa, Homo sapiens
AREEEITPVVSIAYKVLEVFPKGRWVLITCCAPQPPPPITYSLCGTKNIKVAKKVVKTHEPASFNLNVTLKSSPDLLTYFCWASSTSGAHVDSARLQMHWELWSKPVSELRANFTLQDRGAGPRVEMICQASSGSPPITNSLIGKDGQVHLQQRPCHRQPANFSFLPSQTSDWFWCQAANNANVQHSALTVVPPGGDQKMEDWQGPLESPILALPLYRSTRRLSEEEFGGFRIGNGE

SEQ ID NO: 10 - protein, 124 aa, Homo sapiens
VAKKVVKTHEPASFNLNVTLKSSPDLLTYFCWASSTSGAHVDSARLQMHWELWSKPVSELRANFTLQDRGAGPRVEMICQASSGSPPITNSLIGKDGQVHLQQRPCHRQPANFSFLPSQTSDWF

SEQ ID NO: 11 - protein, 237 aa, Homo sapiens
AQYSSDRCSWKGSGLTHEAHRKEVEQVYLRCAAGAVEWMYPTGALIVNLRPNTFSPARHLTVCIRSFTDSSGANIYLEKTGELRLLVPDGDGRPGRVQCFGLEQGGLFVEATPQQDIGRRTTGFQYELVRRHRASDLHELSAPCRPCSDTEVLLAVCTSDFAVRGSIQQVTHEPERQDSAIHLRVSRLYRQKSRVFEPVPEGDGHWQGRVRTLLECGVRPGHGDFLFTGHMHFGEAR

SEQ ID NO: 12 - protein, 95 aa, Homo sapiens
MFGVLEGAQANSENWIAPSGPWALGLWSSLYFLLFSTLEGRGGRVLSQSCSMAVAAASWISRENARSVKRSYMQSSPQRPKEPRNQRTSHTTPVC

SEQ ID NO: 13 - protein, 205 aa, Homo sapiens
SGFTALHWAAKSGDGEMALQLVEVARRSGAPVDVNARSHGGYTPLHLAALHGHEDAAVLLVVRLGAQVHVRDHSGRRAYQYLRPGSSYALRRLLGDPGLRGTTEPDATGGGSGSLAARRPVQVAATILSSTTSAFLGVLADDLMLQDLARGLKKSSSFSKFLSASPMAPRKKTKIRGGLPAFSEISRRPTPGPLAGLVPSFPPTT

SEQ ID NO: 14 - protein, 125 aa, Homo sapiens
SLIVFMEQVHRGIKGLVRDSHGKGIPNAIISVEGINHDIRTANDGDYWRLLNPGEYVVTAKAEGFTASTKNCMVGYDMGATRCDFTLSKTNMARIREIMEKFGKQPVSLPARRLKLRGQKRRQRG

SEQ ID NO: 15 - protein, 99 aa, Homo sapiens
GSLSPTKYNLLELKESCIRNQDCETGCCQRAPDNCESHCAEKGSEGSLCQTQVFFGQYRACPCLRNLTCIYSKNEKWLSIAYGRCQKIGRQKLAKKMFF

SEQ ID NO: 16 - protein, 98 aa, Homo sapiens
SQLELIDLSSNPFHCDCQLLPLHRWLTGLNLRVGATCATPPNARGQRVKAAAAVFEDCPGWAARKAKRTPASRPSARRTPIKGRQCGADKVGHGAGGV

SEQ ID NO: 17 - protein, 112 aa, Homo sapiens
GEAGGSCLRWEPHCQQPLPDRVPSTAILPPRLNGPWISTGCEVRPGPEFLTRAYTFYPSRLFRAHQFYYEDPFCGEPAHSLLVKGKVRLRRASWVTRGATEADYHLHKVGIV

SEQ ID NO: 18 - protein, 64 aa, Homo sapiens
ATKLVTCPAPRQFAVGAFTAAGRAWLFAPSLGASFSKLRSQQRSRDFRGRLFLRAERRAGGFTS

SEQ ID NO: 19 - protein, 237 aa, Homo sapiens
SSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGHSDSDPPYTTLKTCLVAASPREEGMRWALLVLLAFLSPIPAPPTVFCARDHFLVEWDLSFERIFYSFSSGPFPSKAPERKACSQKSSNLEGRMKSVTRPTGSSAEITCDLTVINAVYIHWYLQQEGKTPQHLLHYDV

SEQ ID NO: 20 - protein, 145 aa, Homo sapiens
AGVTQTPKFHVLKTGQSMTLLCAQDMNHEYMYRYRQDPGKGLRLIYYSVAAALTDKGEVPNGYNVSRSNTEDFPLKLESAAPSQTSVYFCASSYSTALQGCLLSAHKGKGRCCPPPPPKTQGCPVQRSLHQEPWNPEWPQVARTV

SEQ ID NO: 21 - protein, 143 aa, Homo sapiens
AKVTQTPGHLVKGKGQKTKMDCTPEKGHTFVYWYQQNQNKEFMLLISFQNEQVLQETEMHKKRFSSQCPKNAPCSLAILSSEPGDTALYLCASSQSTALKCQFLLAHKLVTDPAQEAGDVLGWKGVTENNWSQLKPQCNLTQG

SEQ ID NO: 22 - protein, 50 aa, Homo sapiens
RDKMRMQRIKVCEKRPSIDLCIHHCSYFQKCETNKICCSAFCGNICMSIL

SEQ ID NO: 23 - protein, 55 aa, Homo sapiens
PERWFPGSCHVFGQGHQLFHIFLVLCTLAQLEAVALDYEARRPIYEPLHTHWPHN

SEQ ID NO: 24 - protein, 237 aa, Homo sapiens
QLLMYQQHTSHYDLERKGGYLMLSFIDFCPFSVMRLRSLPSPQRYTRQERYRARPPRVLERSGFHNENSLAIYQGLVYYLLWLHSVYDKPYADPVHDPTWRWWANNKQDQDYYFFLASNWRSAGGVSIEMDSYEKIYNLESAYELPERIFLDKGTEYSFAIFLSAQGHSFRTQSELGTAFQLHSQVDVGVVLADPGCIEASVKQEVLINRNSVLFSITLKDKKLCYDQGISGHHLME

SEQ ID NO: 25 - protein, 140 aa, Homo sapiens
VVEELKLSHNPLKSIPDNAFQSFGRYLETLWLDNTNLEKFSDGAFLGVTTLKHVHLENNRLNQLPSNFPFDSLETLALTNNPWKCTCQLRGLRRWLEAKASRPDATCASPAKFKGQHIRDTDAFRSCKFPTKRSKKAGRH

(19) United States
(12) Patent Application Publication
(10) Pub. No.: US 2014/0106987 A1
(43) Pub. Date: Apr. 17, 2014

(54) BIOMARKERS FOR AUTOIMMUNE LIVER DISEASES AND USES THEREOF

(71) Applicant: ISTITUTO NAZIONALE GENETICA MOLECOLARE - INGM, Milano (MI) (IT)

(72) Inventors: Sergio Abrignani, Rapolano Terme (SI) (IT); Mauro Bombaci, Milano (MI) (IT); Chiara Zingaretti, Ravenna (RA) (IT)

(73) Assignee: ISTITUTO NAZIONALE GENETICA MOLECOLARE - INGM, Milano (MI) (IT)

(21) Appl. No.: 14/125,128

(22) PCT Filed: Jun. 14, 2012
(86) PCT No.: PCT/EP2012/061313

§ 371 Date: Dec. 10, 2013

(30) Foreign Application Priority Data
Jun. 16, 2011 (EP) 11170252.8

(51) Int. Cl. G01N 33/68

(52) U.S. Cl. CPC G01N 33/6893
USPC 506/9; 436/501; 530/324

(57) ABSTRACT

The present invention relates to a method for diagnosis or prognosis of liver autoimmune diseases by means of detecting specific biomarkers in biological samples. The invention refers also to a method of monitoring an autoimmune liver disease pathology status after treatment with surgery and/or therapy in a subject with autoimmune liver disease, to kits and microarrays to perform said methods.

### FIELD OF INVENTION

The present invention relates to the field of immunodiagnostic and/or prognostic of liver autoimmune diseases.

### STATE OF THE ART

Autoimmune Liver Diseases (AILD) are chronic and progressive disorders with a poorly understood etiology. The most common AILD are Autoimmune Hepatitis (AIH) and Primary Biliary Cirrhosis (PBC).

Autoimmune Hepatitis (AIH) is a chronic necro-inflammatory disease and one of the most common autoimmune liver diseases AIH has an incidence of 1-2 per 100,000 per year, and a prevalence of 1-10/100,000. As with most of the other autoimmune diseases, it affects women more often than men (80%), with a sex ratio of about 3:1 (female to male) (Czaja A J. et al., 2010, Gastroenterology; Makol et al., 2011, Hepatitis research and treatment). Histologically it is characterized by: interface hepatitis and plasma cell infiltration; hypergammaglobulinemia is often present; a number of autoantibodies can be detected such as antinuclear antibodies (ANA), anti-Smooth Muscle Antibody (SMA), liver/kidney microsomal antibody (LKM-1), LC1, anti-actin, anti-ASGPR (Bogdanos D P. et al., 2009, Semin Liver Dis). Primary Biliary Cirrhosis (PBC) is a slowly progressing disease causing the destruction of small and medium-size intra-hepatic bile ducts (Selmi C. et al., 2011, Imm Cell Bio). It affects women in 90% of cases. The prevalence is estimated at 0.6-40/100.000. Ursodeoxicholic acid has been shown to improve serum biochemistry, histology and patient's survival (Muratori L. et al., 2010, Dig Liver Dis; Muratori L. et al., 2008, Clin Liver Dis). It is characterized by: anti-mitochondrial antibodies—AMA—(˜90%) and intrahepatic cholestasis (increased alkaline phosphatase—Alk Ph—, normal ultrasonographic—US—scan).

The detection of the AMA autoantibodies is performed routinely by immunofluorescence on fresh multi-organ sections (liver, kidney, stomach) from rodents, but this technique may present many intrinsic problems such as standardization and interpretation of the immuno-morphological patterns (Bogdanos et al., 2008, WJG). To overcome these methodological problems, the International Autoimmune Hepatitis Group established an internationally representative committee to define guidelines and develop procedures and reference standards for more reliable testing (Vergani et al., 2004, Journal of hepatology). In recent years, some AILD target-autoantigens have been identified and characterized (Zachou, K., et al., 2004; J of Autoimm Dis), but little is known on their pathogenetic role, and probably many autoantigens are still unknown. For autoantibodies to have a pathogenetic role, two features have to be met: (i) the autoantigen should be expressed on the target organ and exposed to autoantibodies, (ii) the autoantibodies should have functional activity. Song Q. et al. (2010, J. Proteome Res) described the identification of highly specific biomarkers and their validation for AIH. This study demonstrates that the combination of six autoantigens can be used to diagnose AIH-positive serum samples and that these autoantigens can be effectively used in protein microarray assays, as well as, in traditional ELISA-based assays.

US2009/0023162 discloses the methods for the identification of atypical antineutrophil cytoplasmic antibodies (ANCA), kits suitable for the same and application of said methods to the diagnosis of chronic inflammatory intestinal diseases and autoimmune liver diseases.

RU 2247387 (C1) provides a method involving determination of anti-mitochondrial antibodies, immunoglobulins such as IgA, IgM, IgG, gamma-globulins, anti-gliadin antibodies, and circulating immune complexes for the diagnosis of autoimmune liver injuries in patients with chronic hepatitis.

To date, however, there are no early and precise assays that can be used to identify individuals carrying or at risk of developing AILD. An early diagnosis is clearly important.

Dalekos G. 2002 European J. Int. Medicine, vol. 13, n. 5, pp. 293-303 and Jones D. E. 2000 Journ. Clin. Pathol. Vol. 53, n. 11, pp. 813-821 disclose some autoantigens in AIH and PBC.

### DESCRIPTION OF THE INVENTION

Though the identification of some autoantibodies is within prior art documents, there is still the need to identify novel biomarkers, namely autoantibodies, to diagnose the liver autoimmune disease (AILD) and/or to discriminate between autoimmune and other liver pathologies, and/or to monitor the efficacy of patient treatments of liver autoimmune disease and the disease progression. In the present invention a subject with autoimmune liver disease is named “AILD or hepatic autoimmunity patient” and is affected by autoimmune liver disease, including AIH or PBC.

In the present invention, a panel of 17 autoantigens was identified in patients with AILD by protein array. In addition, 6 out of the 17 autoantigens were also validated by Dissociation Enhanced Lanthanide FluoroImmunoAssay method (DELFIA®), in patients diagnosed with liver autoimmune diseases, and showed individual sensitivities ranging from 42% to 74%. The combined assessment of these six autoantigens displays a 82%±4% sensitivity and almost 90%±3% specificity.

These six autoantigens represent novel markers of liver autoimmunity, such as AIH and PBC. These markers can display much higher sensitivity and specificity (Vs other diseases such as HCV and HBV) when compared to the benchmark markers (CYP2D6 & ASGPR, as described in the Methods section). Therefore, the autoantigens identified in the present invention are valuable tools for the development of a new serological assay that is easy to perform and is highly specific for AILD diseases. This assay could significantly contribute to an improvement of AILD diagnosis and to a discrimination between PBC and AIH.

It is therefore an object of the present invention an in vitro method of diagnosis or prognosis or evaluation of risk to develop a liver autoimmune disorder belonging to the group of AIH and PBC in a subject, comprising the steps of:

a) contacting a biological sample from the subject with a protein comprised in the group of: a protein having the amino acid sequence SEQ ID No. 1, an allelic variant, an orthologous, at least one immunological fragment or a functional equivalent thereof, under conditions appropriate for binding of autoantibodies, if present in the biological sample, to said protein, and
b) detecting the presence of bound autoantibodies.

In the context of the instant invention the term “protein” includes:
i. the whole protein, allelic variants and orthologous thereof;
ii. any immunological, synthetic or recombinant or proteolytic fragment of a protein having the ability to be recognized by and bound to antibodies directed against the protein;
iii. any functional equivalent comprised in the group of synthetic or recombinant functional analogs having the ability to be recognized by and bound to antibodies directed against the protein.

In a preferred embodiment, step a) is performed by contacting said biological sample with the protein having the amino acid sequences SEQ ID No. 1 and at least one further protein selected from the group of 16 proteins having the amino acid sequences SEQ ID No. 2, 3, 5, 6, 7, 8, 9, 10, 11, 12, 14, 15, 16, 17, 22, 25, allelic variants, orthologous, immunological fragments or functional equivalents thereof.

In a further embodiment step a) is performed by contacting said biological sample with three proteins having the amino acid sequences SEQ ID No. 1, 11, 17 allelic variants, orthologous, immunological fragments or functional equivalents thereof.

In a further preferred embodiment step a) is performed by contacting said biological sample with four proteins having the amino acid sequences SEQ ID No 1, 10, 11, 17, allelic variants, orthologous, immunological fragments or functional equivalents thereof.

In a further preferred embodiment step a) is performed by contacting said biological sample with six proteins having the amino acid sequences SEQ ID No 1, 6, 8, 10, 11, 17 allelic variants, orthologous, immunological fragments or functional equivalents thereof.

Preferably the biological sample is comprised in the group of blood, serum, plasma, urine, saliva, mucus, or fractions thereof.

Preferably the biological sample is from an adult or from an adolescent.

Preferably the detection of said bound autoantibodies is performed by means of binding to specific ligands. More preferably the ligands are conjugated with detecting means.

It is another object of the invention a method of monitoring an autoimmune liver disorder after treatment with surgery and/or therapy in a subject with said autoimmune liver disorder, comprising the step of following the modulation of antibodies as disclosed.

In a preferred aspect said proteins or immunological fragments or functional equivalents thereof are displayed on one or more protein microarrays.

It is another object of the invention the use of a protein microarray comprising at least the proteins as defined or immunological fragments or functional equivalents thereof for performing the method according to the invention.

It is another object of the invention the use of a solid support for an immunodiagnostic assay comprising at least the proteins as defined or immunological fragments or functional equivalents thereof for performing the method according to the invention.

It is another object of the invention the use of an immunodiagnostic kit comprising the above solid support and detecting means for performing the method according to the invention.

In the present invention a subject with autoimmune liver disease is named “AILD or hepatic autoimmunity patient” and is affected by any autoimmune liver disease, as AIH or PBC.

HCV patients are patients diagnosed with hepatitis C and displaying autoreactive antibodies, or patients with HCV infection or patients with hepatitis C and non hepatic autoimmune diseases such as crioglobulinemia or thyroid dysfunctions.

### DETAILED DESCRIPTION OF THE INVENTION

The invention will be now described by means of non limiting examples referring to the following figures.

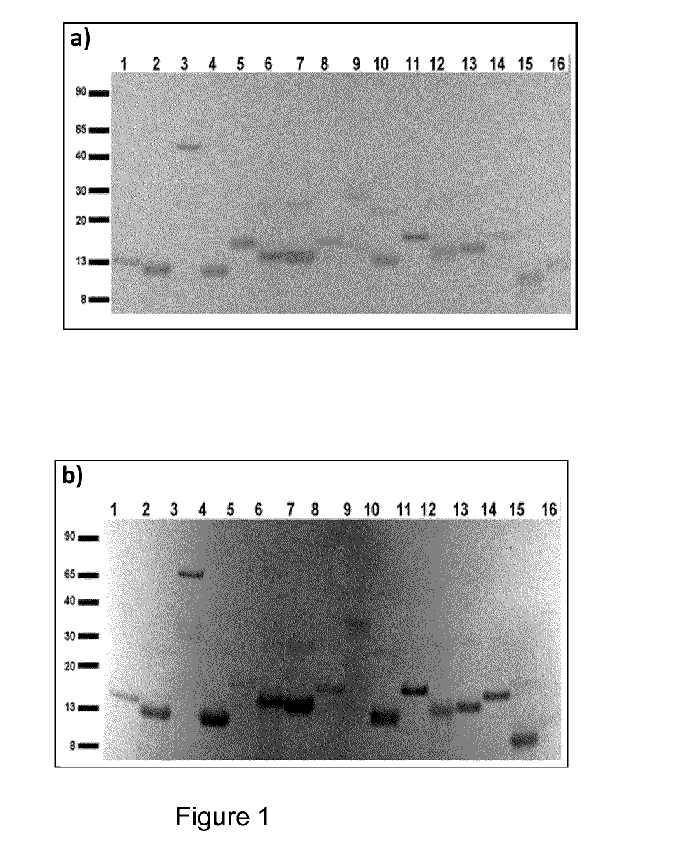

FIG. 1: a) SDS-PAGE of a representative set of purified recombinant human proteins stained with Coomassie blue; b) Western Blot analysis using an anti-histidine (His) tag monoclonal Antibody (mAb); molecular weight markers are shown on the left in kDa.

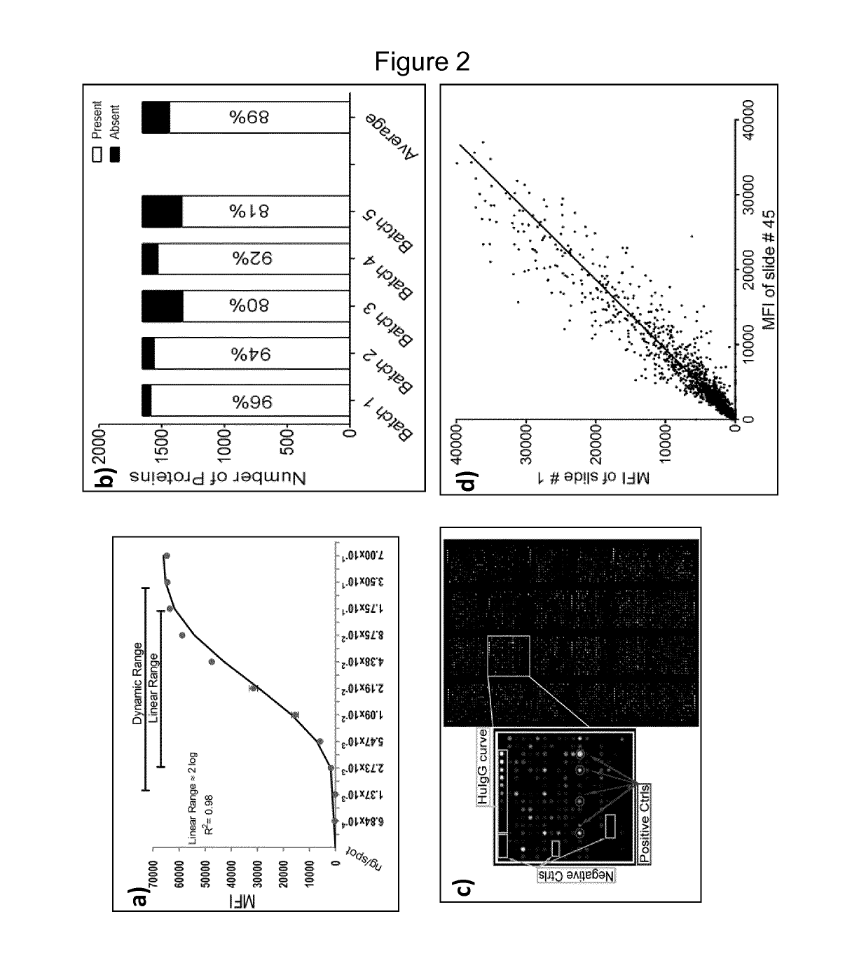

FIG. 2: a) Plot of the control human IgG curve. The dots correspond to the experimental average signal (Mean Fluorescence Intensity, y-axis) detected for each IgG concentration reported on the x-axis in nanograms per spot (ng/spot), while the continuous line corresponds to the interpolated resulting sigmoid curve. Dynamic Range: refers to a range of values that can be measured by a signal (i.e. Mean Fluorescence Intensity). Linear Range: is the range of concentration over which the signal intensity is linear, i.e. directly proportional to the spot concentration. b) Number of proteins detected with the anti-His mAb. About 90% of proteins were spotted with success on the slides (i.e. about 90% of the proteins produced signals that were significantly above the background signal). Histograms: Proteins were considered “Present” when at least two out of the four replicates gave a signal above the background, otherwise they were considered “Absent”. c) The human protein microarray containing 1626 His-tagged recombinant proteins was probed with an anti-His mAb followed by a secondary α-mouse antibody Alexa-647-labeled. The whole microarray included 24 grids (as the one shown in the enlarged box) each one containing 304 proteins spots and including also positive (such as viral and bacterial proteins) and negative (such buffer or BSA) controls (positive Ctr and negative Ctr frames), as well as IgG calibration curve (HulgG curve) for data normalization. Proteins and controls were always spotted in quadruplicates, while IgG were spotted in eight replicates. d) Correlation among spot intensities of two different slides (Slide 1 Vs Slide 45) of the same batch. The scatter plot indicates a positive correlation. The correlation coefficient is 0.9, indicating a high reproducibility of the signals derived from the proteins spotted.

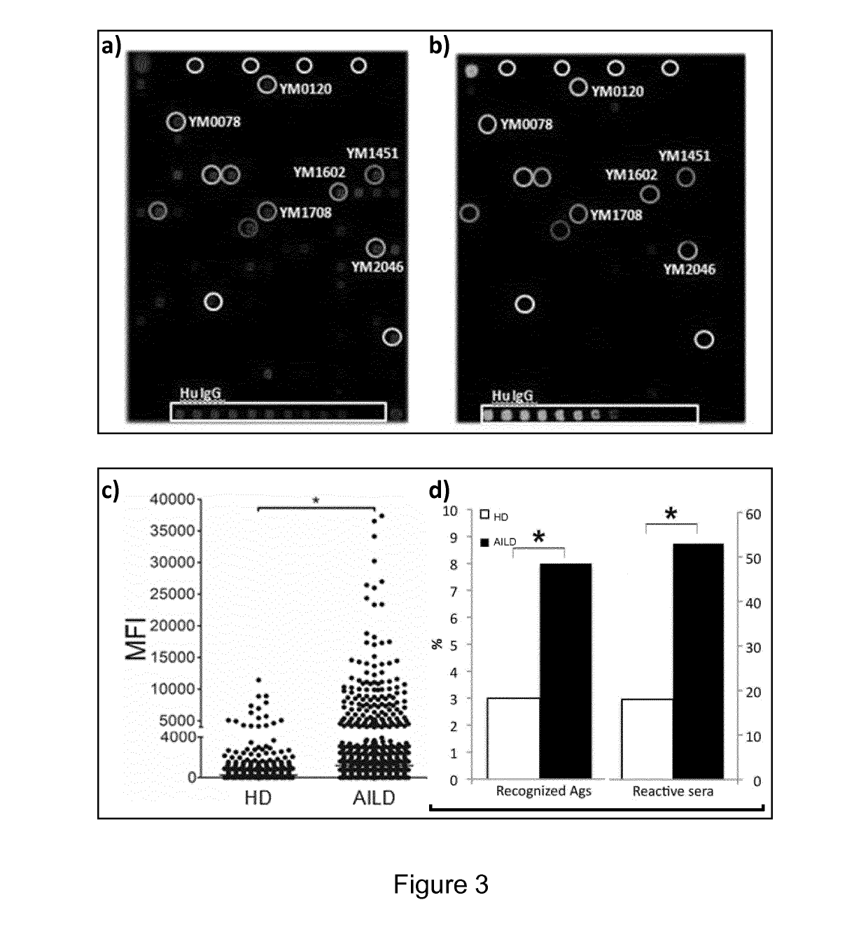

FIG. 3: Differences in immunoreactivity between serum samples. Representative image of the autoantigen microarrays incubated with a) sera from an AILD patient and b) a healthy donor (HD). c) Comparison of Mean Fluorescence Intensities (MFIs) of all spotted proteins against two sera populations: Healthy Donor (HD), Autoimmune Liver Diseases (AILD) patients. Each dot represents the MFI of a single protein. A cut-off value 4.000 was used to score a protein as positive. Asterisks: statistical significance, Student's t-test (pval <0.01). d) Percentage of antigens recognized by more than 15% of the sera tested; the threshold was determined on the basis of the average HD recognition (left bars), and the percentage of reactive sera for each population (i.e. sera reacting with more than 3% of the proteins screened; the threshold was determined on the basis of the average HD reactivity) (right bars). Asterisk; statistical significance (χ² test (pval <0.01).

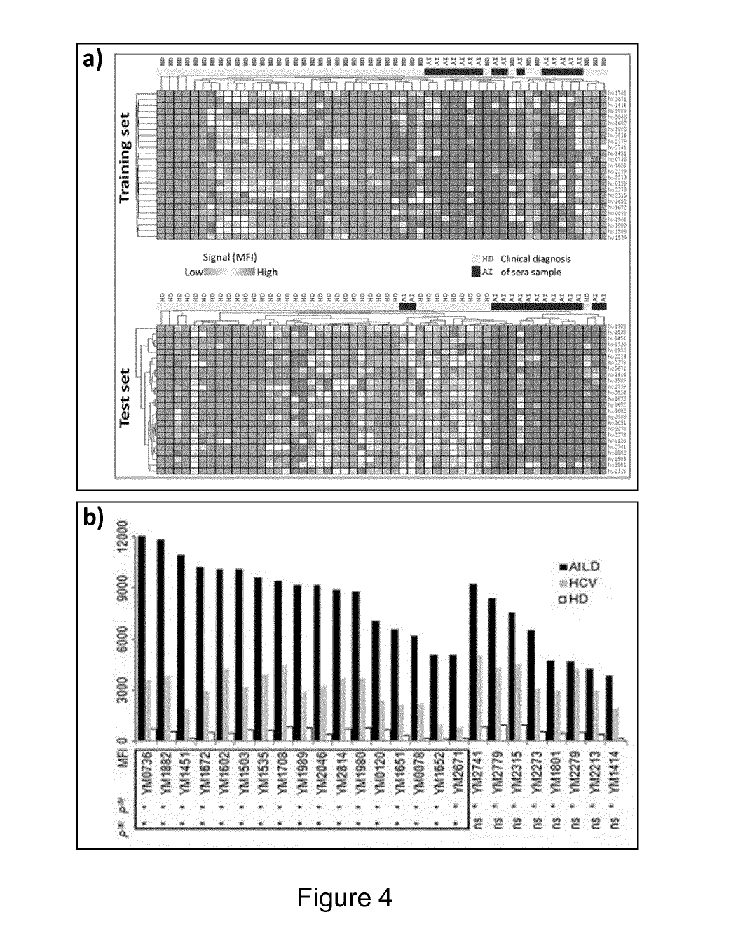

FIG. 4: a) Hierarchical Clustering of Training (upper panel) and Test set (lower panel) with regard to the 25 autoantigens selected as specifically recognized by AILD patients on the basis of i) higher recognition frequency and ii) higher mean fluorescence intensities as compared to healthy donors. Sera are represented in columns while proteins are in rows. Red indicates positive immunoreactivity, and blue low or no immunoreactivity; b) Bar graph of Mean Fluorescence intensities of the 25 autoantigens selected in Discovery sample set (Training and Test set). Significant differences were observed between AILD and HCV patients sera for seventeen autoantigens (into the box). Asterisks, p val <0.01 (Student's t-test). P^{(a)}: p-val of AILD versus Hepatitis C patients, P^{(b)}: p-val of AILD versus Healthy donors.

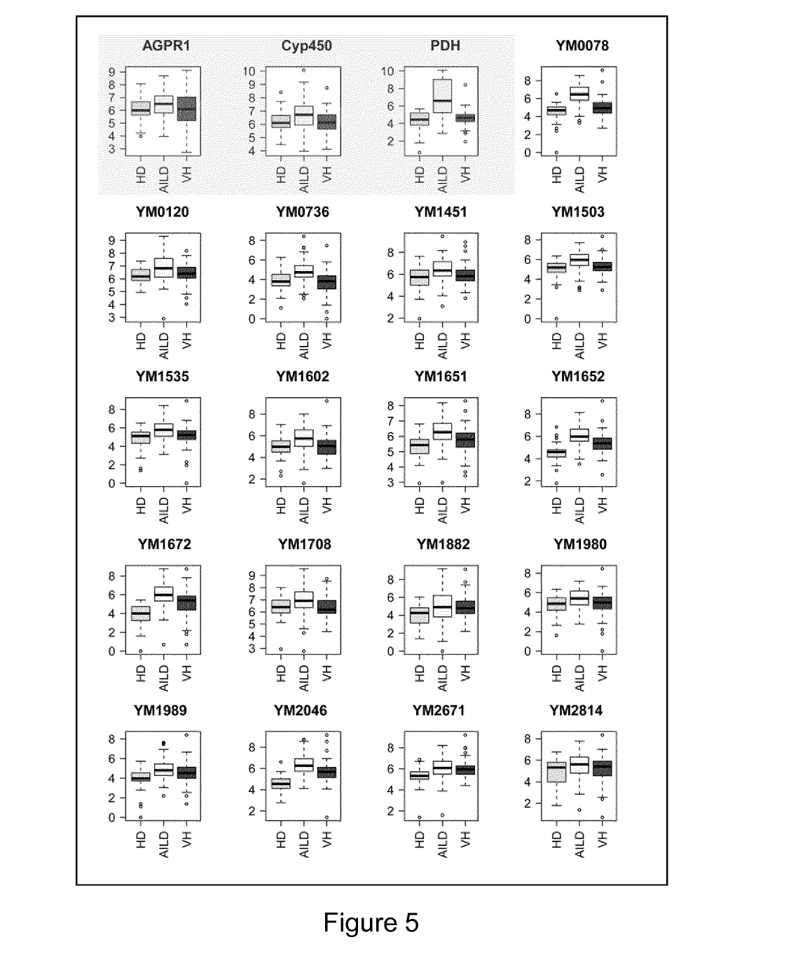

FIG. 5: Boxplots of DELFIA results for the 17 autoantigens tested. The signal distributions, after natural logarithm trasformation, are displayed. The boxes define the interquartile range (IQR). The extreme outliers beyond the 1.5 IQR+median are showed as dots. Three known AILD control proteins (AGPR-1, CYP2D6, PDH) (grey box) were also compared. HD: Helathy donors; AILD: autoimmune liver disease, VH: Viral hepatitis.

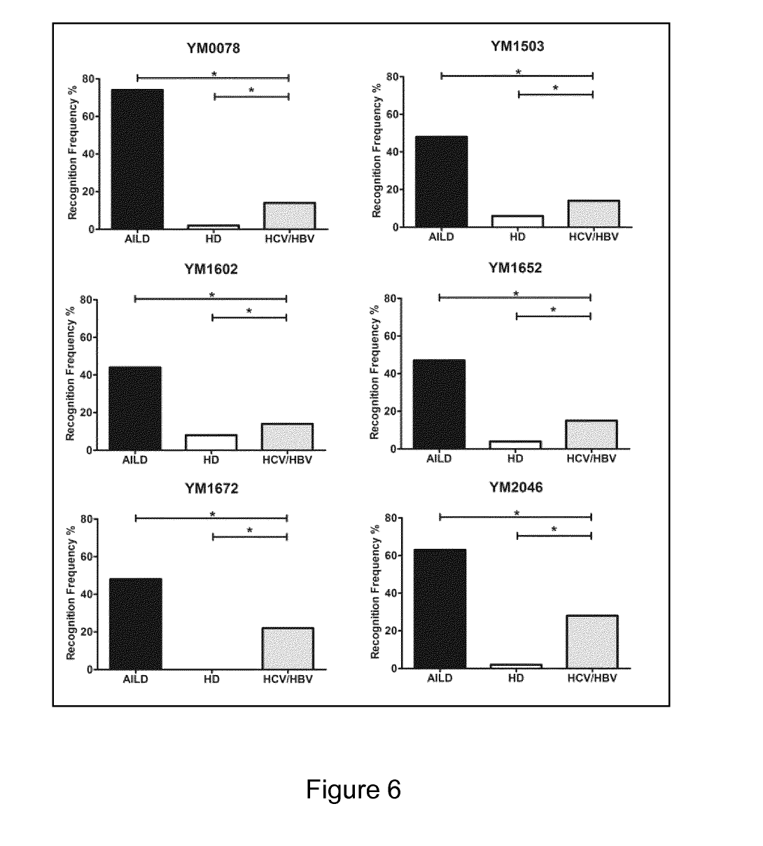

FIG. 6: Six proteins are confirmed as AIH autoantigens in DELFIA® assay. Recognition frequency of the best six autoantigens as determined by DELFIA®. Proteins were tested with sera from AILD patients (n. 50 AIH and n. 50 PBC), healthy donors (n. 50) and HBV or HCV viral hepatitis (VH) patients (n. 74). Each sera was tested 3 times in independent experiments. Asterisk: statistical significance (Fisher exact test, pval <0.01).

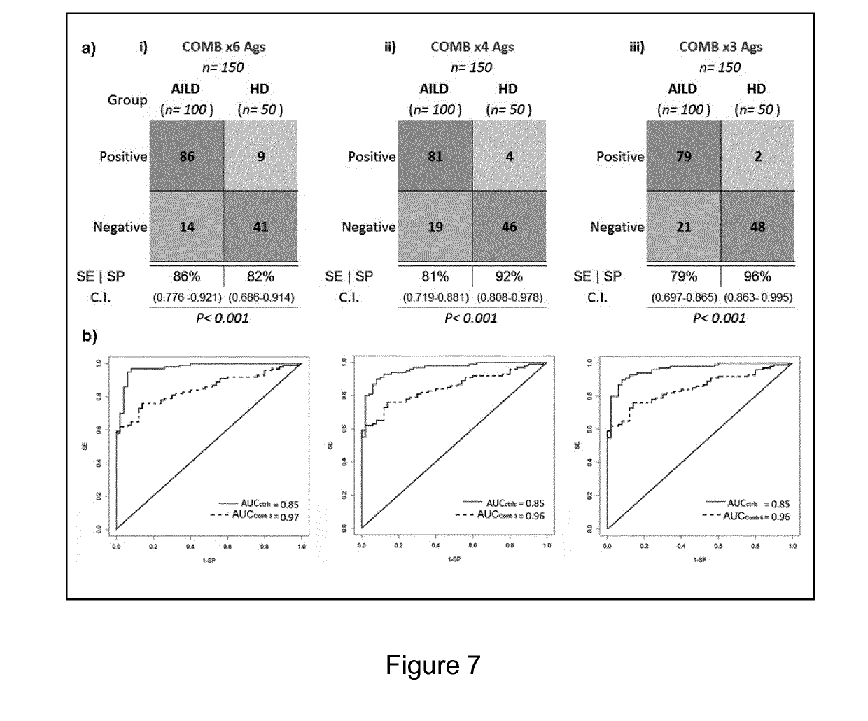

FIG. 7: Combination of the six autoantigens identifies AILD patients with high sensitivity and specificity. (a) Numbers in the boxes indicate AIH and HD sera that, in DELFIA® assay, recognize (positive) or do not recognize (negative) combination of six (i), four (ii) or three (iii) of the six autoantigens. Sensitivity (SE) and Specificity (SP) with 95% confidence intervals (C.I.) are indicated in the lower part of the panels. p-values are calculated with χ² tests. (b) Logistic regression models for the six, four and three autoantigens (red curves) and the three known control proteins (AGPR1, CYP2D6, PDH) (black curves) were calculated and represented as ROC curves and corresponding values of Area Under the Curve (AUC).

### MATERIAL AND METHODS

### Serum Samples

Samples used for this study were collected in five different hospitals: i) Policlinico Ospedale Maggiore, Transfusional Unit, Milan; ii) Hepatology Unit, University Hospital, Pisa, Italy; iii) Sant'Orsola-Malpighi University Hospital, Bologna, Italy; iv) Center for Autoimmune Liver Diseases, IRCCS Istituto Clinico Humanitas, Rozzano, Italy; v) Center for Systemic Manifestations of Hepatitis Viruses (MaSVE), University of Firenze, Italy. For the discovery phase, 218 sera were used (15 AIH, 15 PBC 78 HD, 110 HCV), while for the validation phase 224 sera were used (50 AIH, 50 PBC, 50 HD, 50 HCV, 24 HBV). Table 1 reports the clinical characteristics and ages of the patients and donors enrolled in this invention, for the microarray analysis (Example 2-3).

TABLE 1
Clinical characteristics of the serum samples used in this invention

| | | | | | | | Age |
|---|---|---|---|---|---|---|---|
| | | | Sub | | | Mean ± SD | Sex |
| | | Group | group^{\hat{ }} | n. | Source* | (median) | Gen (n.) |
| Discovery | Training | Healthy Donors | HD | 39 | 1 | 44 ± 10 | f(12) |
| | Set | | | | | (46) | m(27) |
| | | Liver Autoimmune | AIH | 8 | 2 | 51 ± 20 | f(8) |
| | | disease | | | | (52) | m(0) |
| | | | PBC | 7 | 2 | 50 ± 12 | f(6) |
| | | | | | | (52) | m(1) |
| | Test Set | Healthy Donors | HD | 39 | 1, 2 | 44 ± 10 | f(8) |
| | | | | | | (44) | m(31) |
| | | Liver Autoimmune | AIH | 7 | 2 | 49 ± 23 | f(5) |
| | | disease | | | | (54) | m(2) |
| | | | PBC | 8 | 2 | 59 ± 22 | f(8) |
| | | | | | | (56) | m(0) |
| | | Viral hepatitis | HCV | 110 | 2, 3 | 55 ± 15 | f(41) |
| | | | | | | (56) | m(69) |

| | | | | | | |
|---|---|---|---|---|---|---|
| Validation | Healthy Donors | HD | 50 | 1 | 45 ± 9 | f(8) |
| | | | | | (47) | m(42) |
| | Liver Autoimmune disease | AIH | 50 | 2, 4 | 45 ± 21 | f(41) |
| | | | | | (49) | m(9) |
| | | PBC | 50 | 2, 4 | nd ± nd (nd) | f(nd) |
| | | | | | | m(nd) |
| | Viral hepatitis | HCV | 50 | 5 | 52 ± 20 | f(20) |
| | | | | | (52) | m(30) |
| | | HBV | 24 | 2 | 51 ± 14 | f(8) |
| | | | | | (52) | m(16) |

*Origin of samples: (1) Transfusional Unit, Ospedale Maggiore Policlinico, Milan; (2) Sant'Orsola University Hospital, Bologna; (3) Hepatology Unit, University Hospital, Pisa; (4) Center for Autoimmune Liver Diseases, IRCCS Istituto Clinico Humanitas, Rozzano; (5) Center for Systemic Manifestations of Hepatitis Viruses (MaSVE), Firenze.
\hat{ }HD: Healthy Donor; AIH: Autoimmune hepatitis; PBC: Primary Billiary Cirrhosis; HCV: Chronic C viral hepatitis patients; HBV:Chronic B viral hepatitis patients

### Human Proteins: Selection, Expression and Purification

Genes whose translated products carry a secretion signal peptide or at least one transmembrane domain were selected, cloned and expressed in a high through-put system as histidine-tagged products as described (Grifantini R. et al., 2011, Journal of proteomics). 1626 full-length proteins or protein domains were expressed in E. coli and purified from the bacterial insoluble fraction by Immobilized metal ion affinity chromatography (IMAC, GE).

Human, viral or bacterial proteins were used as biological or technical controls in the microarray. In particular genes encoding for Core protein and Non-structural proteins NS3 (from HCV genotype 1), NS3-4a (from HCV genotype 2) NS5b (from HCV genotype 1), Tetanus toxin and H1N1 were subcloned in E. coli strain DH5α and expressed in BL21(DE3), respectively. Bovine Serum Albumin (BSA), Human Serum Albumin, Human Glutathione-S-Transferase and Protein A from Staphylococcus aureus were purchased from Sigma.

For DELFIA® experiments, Pyruvate DeHydrogenase (PDH) protein was purchased by Sigma, while genes encoding Cytochrome P450 2D6 (CYP2D6) and asialoglycoprotein receptor 1 (ASGR-1) from Ultimate™ Human ORF Clones were purchased by Invitrogen and were subcloned in E. coli strain DH5α and expressed in BL21(DE3), respectively. All the corresponding proteins were purified by affinity chromatography on IMAC resin.

### Protein Quality Control

Purified recombinant proteins (10-15 μg total protein), obtained as described above, were stored at 4° C. and analyzed by SDS-PAGE (Criterion PAGE system Bio-Rad) followed by Coomassie Blue staining of the gel immediately before spotting them, to assess their integrity, and purity level. Proteins showing purity levels >70% (ChemiDoc™ XRS, Quantity One® software; Bio-Rad) were used for protein array preparation.

For Western blot analysis, aliquots (0.5 μg) of the proteins were resolved on 4-12% pre-cast SDS-PAGE gradient Tricine gels under reducing conditions, and electroblotted onto nitrocellulose membranes (Bio-Rad), according to the manufacturer's instructions. The membranes were blocked with 5% non-fat milk in 1×PBS plus 0.1% Tween 20 (TPBS) for 1 h at room temperature, incubated with the α-His mAb (GE-Healthcare) diluted 1:1000 in 3% non-fat milk in TPBS 0.1% for 1 h at room temperature, and washed three times in TPBS 0.1% (FIG. 1). The secondary HRP-conjugated antibody (α-mouse immunoglobulin/HRP, GE-Healthcare) was diluted 1:1000 in 3% non-fat milk in TPBS-0.1% and incubated for 1 h at room temperature. The proteins were visualized by enhanced chemiluminescence (Super Signal West Pico Chemiluminescence Substrate Thermo Scientific, USA) according to the manufacturer's specifications. Chemiluminescence was detected with an LAS-3000 (Fujifilm, USA).

### Protein Microarray Printing

Protein MicroArrays were generated by spotting the 1626 affinity-purified recombinant proteins (0.5 mg/ml, in 6M Urea) in 4 replicates on nitrocellulose-coated slides (FAST slides, GE-Healthcare) using Stealth SMP3® spotting pins (TeleChem International, Sunnyvale, Calif.) and a Microgrid II microarray contact printer (Biorobotics), resulting in spots of approximately 130 μm in diameter. As experimental positive control, to assess the sensitivity and reproducibility of the arrays and for signal normalization, a curve of human IgG(s) at 11 different concentrations (solutions from 0.001 to 1 mg/ml) was spotted on the arrays in 8 replicates (in 6M Urea) and detected with Alexa-647 conjugated α-Human IgG secondary antibody (Invitrogen). As negative controls the spotting buffer alone, was printed and used to assess possible non-specific signals due to cross contamination.

A quality control of the spotting procedure was performed on 10% of randomly chosen slides, by confirming the presence of the total immobilized proteins using the α-His mAb, followed by detection using a Alexa-647 conjugated α-Human IgG secondary antibody (FIG. 2). The spotted microarrays were allowed to remain at room temperature for 1 h before storage at 4° C. until use.

### Incubation and Scanning of Protein Microarray

Incubation was automatically performed with a TECAN Hybridization Station (HS⁴⁸⁰⁰™ Pro; TECAN, Salzburg, Austria). The microarray slides were prewashed 3 min in TPBS 0.1% Tween 20, and saturated with BlockIt™ Microarray Blocking Buffer (Arrayit Corporation) for 45 min at 25° C. under mild agitation. After injection of 105 μl of individual serum (diluted 1:300 in Blocking Buffer plus 0.1% Tween 20), incubation was performed at 25° C. for 45 min with low agitation. The microarrays were washed at 25° C. in TPBS for three cycles of 1 min wash time and 30 sec soak time.

Afterwards the microarray slides were incubated at 25° C. for 1 with Alexa-647 conjugated α-human IgG (Invitrogen) diluted at 1:800 in Blocking Buffer in the dark. The microarrays were again washed at 25° C. in TPBS for two cycles of 1 min wash time and 30 sec soak time, in PBS for two cycles of 1 min: 30 sec and finally in milliQ sterile water for one cycle of 15 sec.

The microarray slides were finally dried at 30° C. under nitrogen for 2 min, and scanned using a ScanArray Gx PLUS (PerkinElmer, Bridgeport Avenue Shelton, USA). 16-bit images were generated with ScanArray™ software at 10 μm per pixel resolution and processed using ImaGene 8.0 software (Biodiscovery Inc, CA, USA). Laser of 635 nm was used to excite Alexa-647 dye. The fluorescence intensity of each spot was measured, signal-to-local-background ratios were calculated by ImaGene, and spot morphology and deviation from the expected spot position were considered using the default ImaGene settings.

### Data Analysis

For each sample, the Mean Fluorescence Intensity (MFI) of replicated spots was determined, after subtraction of the background value for each spot, and subsequently normalized on the basis of the human IgG curve to allow comparison of data from different set of experiments (Bombaci M. et al., 2009, PLoS One). Briefly, the MFIs values of IgG, spotted at different concentrations, were best fitted by a sigmoid curve, using a maximum likelihood estimator (Harris J W et al., 1998, Handbook of Mathematics and Computational Science). The experimental average IgG curve of each slide was adjusted on the reference sigmoid IgG curve, and the background-subtracted MFI values of each protein were normalized accordingly. On the basis of these results, a normalized MFI value of 4.000 was chosen as the lowest signal threshold for scoring a protein as positively recognized by human sera. For each protein, a Coefficient of Variation (CV %), was calculated on the four replicate spots, for intra-assay reproducibility (Bombaci et al., 2009, PLoS One).

Recognition Frequency was defined as the percentage of sera reacting with a particular antigen in protein array with a MFI 4.000, and it was calculated for each group of sera. TIGR Multiexperiment Viewer (version MeV4.5) software (Saeed, A. I., et al., 2006, Methods in enzymology) was used to perform an unsupervised bi-dimensional hierarchical clustering.

### Dissociation-Enhanced Lanthanide Fluorescence ImmunoAssay DELFIA® Assays

The DELFIA® assay is a time-resolved fluorescence method that can be used to study antibody binding to solid-phase proteins or peptides. The purified recombinant proteins were used at a concentration of 20 μg per milliliter (Frulloni L. et al., 2009, N Engl J Med) in 6 M urea to coat DELFIA® plates (PerkinElmer). Plates were then blocked for 1 hour at 37° C. with a blocking reagent (PerkinElmer). The blocking buffer was than discarded, and the serum samples were diluted in a 1:300 solution in phosphate buffered saline plus 1% bovine serum albumin (Sigma), plus 0.1% Tween 20 (Sigma) and incubated on the plates 1 hour at 37° C. Plates were then washed 5 times with washing buffer (PerkinElmer). Bound antibodies were detected with europium-labeled α-human IgG serum (1:500 in diluting buffer, PerkinElmer), incubated 30 min at RT in the dark. The wells were again washed in the same washing buffer. After a 10 min incubation at RT, the plates were read on a Infinite F200 PRO instrument (Tecan). Fluorescence intensity values higher than the mean of buffer plus 3 standard deviations were considered to be positive.

### Statistical Analysis

Results of Protein Microarray and DELFIA® experiments from sera of patients and healthy donors were compared using the two-tailed χ² test, the Student's t-test or the Fisher's exact tests. The ANOVA test was used for all the others analyses. The Benjamini-Hochberg correction for multiple testing was used for the analysis of microarray data. Statistical analysis was carried out with the use of GraphPad Prism 5 software, version 5.01. To evaluate the performance of autoantigens combinations in discriminating AILD patients from Healthy donors or HCV patients, logistic regression analysis was performed with R. We the signals of respectively 6, 4 or 3 selected autoantigens we created logistic regression models. The probabilities were calculated as follows: p=exp((Σ(bᵢxᵢ)+c)/(1+Σ(bᵢxᵢ)+c), where p is the probability of each case, i=1 to n; b is the regression coefficient of a given autoantigen, x is signal intensity and c is a constant generated by the model. ROCR package was used to obtain the ROC curves of the models and the Area Under Curve (AUC) values (Sing T. et al. 2005, Bioinformatics).

### EXAMPLES

### Example 1

### Design and Development of a Human Protein Microarray

To study the serological profile of patients diagnosed with autoimmune liver diseases versus a panel of self proteins, the authors developed a protein array by printing 1626 human recombinant (see details in Materials and Methods section) products that corresponded to 1371 distinct human proteins distributed as shown in Table 2.

TABLE 2
Predicted subcellular localization of the human recombinant proteins
for the microarray construction (Grifantini et al., 2011).

| Compartment | % of proteins |
|---|---|
| Cell membrane | 46 |
| Secreted | 23 |
| Intracell. Membrane | 11 |
| Cytoplasm | 11 |
| Mito. Membrane | 4 |
| Nucleus | 4 |
| Mithocondrion | 1 |

Briefly, 1329 of the 1371 the proteins were first selected through a bioinformatic analysis of the whole human genome as translated sequences carrying i) signal peptides, ii) at least one transmembrane domain, iii) having unknown biological function. Fortytwo of the 1371 proteins had a well known immunological function, CD number assigned and were all surface exposed. The majority of printed human proteins were expressed as N-terminal His-tag fusions while 48 proteins where expressed as double tagged fusions, with an N-terminal glutathione S-transferase (GST) and with C-terminal Histidines. Proteins obtained after affinity purification from the bacterial insoluble fraction showed purity levels >70%, as estimated by densitometric scan of SDS-PAGE gels (see Materials and Methods) (FIG. 1a). Moreover, we performed western blot analysis on random protein samples with the anti-His mAb. About 80% of the probed proteins gave a positive signal. In addition for almost 90% of the latter proteins, the molecular weights of the bands detected by western blot corresponded to those observed by SDS-PAGE (FIG. 1b).

Protein arrays were prepared by printing onto nitrocellulose-covered glass slides four replicates of each protein. In addition, we included in the array, several biological and technical controls including human, viral and bacterial proteins (Materials and Methods). Replicates were randomly distributed to get optimal signal reproducibility. Moreover. eleven different amounts of human IgGs at a known concentration (from 8.24×10⁻⁴ to 7×10⁻¹ ng of immobilized protein/spot) were printed also on the array in eight replicates (FIG. 2a).

The quality of the immobilized proteins on the arrays were determined by probing 10% of the slides with an anti-His mAb, and 89% of the proteins produced signals that were significantly above the background (FIG. 2b). The final protein array design consisted of 24 grids each of 304 spots, for a total of 7296 spots (FIG. 2c). The correlation among spot intensities of two different slides of the same batch indicates a high reproducibility of the signals derived from the proteins spotted (FIG. 2d).

### Example 2

### Patients Affected by Autoimmune Liver Diseases Show Higher Auto-Immunoreactivity as Compared to Healthy Donors

In an attempt to determine a panel of autoantigens differentially recognized by patients sera with AILD compared to healthy individuals (HD), the protein microarrays were probed with a sample set (defined as Training set as indicated in Table 1 in Materials and Methods) comprising 15 sera patients with AILD, and 39 sera from healthy donors. The clinical characteristics of each group of sera are summarized in Table 1.

Sera reactivity was evaluated by detecting total IgG bound to each protein spot using Alexa-647 conjugated α-human IgG and measuring the fluorescence intensity (FI) values for each protein. To compare data from different experiments, we used a normalization method, as previously described (Bombaci M et al., 2009, PLoS One). Briefly, the experimental average IgG curve of each slide was adjusted on a reference sigmoid IgG curve, and the background-subtracted mean fluorescence intensities (MFIs) values of each protein were normalized accordingly. On the basis of these results, a normalized-MFI value of 4000 was chosen as the lowest signal threshold for scoring a protein as positively recognized by human sera.

First of all, total reactivity of patients against healthy donors sera was evaluated. Representative images of a zoomed grid of the microarray probed with sera derived from healthy donors and AILD patients are shown in FIGS. 3a and b. As depicted in FIG. 3c, the strongest autoreactivity was observed against all the proteins present on the arrays in patients sera with AILD, but not in the control sera (HD group).

Significant divergences between patients and healthy donors were also observed in terms of the recognition frequencies, indicating differential protein-specific IgG levels FIG. 3d: in details, the percentage of proteins recognized by more than 15% of the tested sera with MFI>4000 was about 8% (132 proteins) in the case of both patients groups but only 3% (41 proteins) in the case of healthy donors (left). On the other hand, 53% of patients with AILD contain antibodies that react intensively with at least 3% of the proteins on the arrays, whereas only 18% of 39 controls had such antibodies (right panel). In both cases the differences observed between the reactivity of patients and healthy donors were statistically significant (chi-square test, p val <0.01).

### Example 3

### Selection and Identification of Specifically Recognized Autoantigens Profile

Having previously established that patients with AILD of Training set displayed an increased autoreactivity when compared to healthy donors, the authors selected the autoantigens specifically recognized by those patients. Following normalization, individual autoantigens from protein microarray were ranked according to (i) the recognition frequency and (ii) the mean fluorescence intensity of AILD patients as compared to the healthy donors.

To be considered of potential interest, the antigen-specific responses had to occur with a significantly higher signal intensity in patients sera than in healthy donors sera (T test's p val <0.01). In particular, the proteins should be recognized in less than 10% of the healthy donors sera and in at least 25% of sera from patients groups (Fisher test's pval <0.01). By using this approach, the authors identified 25 distinct proteins (Table 3) showing a higher immunoreactivity with AILD patients .compared to controls sera.

TABLE 3
Brief description of the human sequences listed

| SEQ | | | |
|---|---|---|---|
| ID | | Accession | |
| NO | Prot_ID | Protein | Amino acid sequence |
| NO: 1 | YM0078 | ENSP00000379111 | PRAPGNLTVHTNVSDTLLLTWSNPYPPDNYLYNHLTYAVNIWSEN |
| | | | DPADFRIYNVTYLEPSLRIAASTLKSGISYRARVRAWAQCYNTTW |
| | | | SEWSPST |
| NO: 2 | YM0120 | ENSP00000386923 | SNWGCYGNIQSLDTPGASCGIGRRHGLNYCGVRASERLAEIDMP |
| | | | YLLKYQPMMQTIGQKYCMDPAVIAGVLSRKSPGDKILVNMGDRTS |
| | | | MVQDPGSQAPTSWISESQVSQTTEVLTTRIKEIQRRFPTWTPDQY |
| | | | LRGGLCAYSGGAGYVRSSQDLSCDFCNDVLARAKYLKRHGF |
| NO: 3 | YM0736 | ENSP00000350556 | MFVDNRIQKSMLLDLNKEIMNELGVTVVGDIIAILKHAKVVHRQDM |
| | | | CKAATESVPCSPSPLAGEIRRGTSAASRMITNSLNHDSPPSTPPR |
| | | | RPDTSTSKISVTVSNKMAAKSAKATAALARREEESLAVPAKRRRV |
| | | | TAEMEGKYVINMPKGTTPRTRKILEQQQAAKGLHRTSVFDRLGAE |
| | | | TKADTTTGSKPTGVFSRLGATPETDEDLAWDSDNDSSSSVLQYA |
| | | | GVLKKLGRGPAK |
| NO: 4 | YM1414 | ENSP00000404259 | SQGVCSKQTLVVPLHYNESYSQPVYKPYLTLCAGRRICSTYRTMY |
| | | | RVMWREVRREVQQTHAVCCQGWKKRHPGALTCEA |
| NO: 5 | YM1451 | ENSP00000343084 | HEAHKTSLSSWKHDQDWANVSNMTFSNGKLRVKGIYYRNADICS |
| | | | RHRVTSAGLTLQDLQLWCNLRSVARGQIPSTL |
| NO: 6 | YM1503 | ENSP00000375259 | GVAEFHMSLTVSCPDPTPSTDPQGRHNREPILGRDDDFMCKQVK |
| | | | FRMCVVGRDGNAQSSVRYTGPLYRRKIRTEFLFVVFLLETRELKP |
| | | | QVNKNVQGTRPS |
| NO: 7 | YM1535 | ENSP00000288466 | MVFVLTYMDPKGEVKKTHLHLASFSPSSEVSCFTNKAQAKNCSV |
| | | | EGCPSEWSSPRNLRSTKSIGTIRATGGCLCSGTVLHFPIPGSASQ |
| | | | ASL |
| NO: 8 | YM1602 | XP_934154 | ATGILICMTKNLESVHSIVLAHSCYHHENKPRPDCCFQQKIRDTKS |
| | | | KVELPRHAHARLTNPQLTHRSMKINDCCIKPLRFGVTCYAAFCDN |
| | | | N |
| NO: 9 | YM1651 | ENSP00000343493 | AREEEITPVVSIAYKVLEVFPKGRWVLITCCAPQPPPPITYSLCGTK |
| | | | NIKVAKKVVKTHEPASFNLNVTLKSSPDLLTYFCWASSTSGAHVD |
| | | | SARLQMHWELWSKPVSELRANFTLQDRGAGPRVEMICQASSGS |
| | | | PPITNSLIGKDGQVHLQQRPCHRQPANFSFLPSQTSDWFWCQAA |
| | | | NNANVQHSALTVVPPGGDQKMEDWQGPLESPILALPLYRSTRRL |
| | | | SEEEFGGFRIGNGE |
| NO: 10 | YM1652 | ENSP00000413076 | VAKKVVKTHEPASFNLNVTLKSSPDLLTYFCWASSTSGAHVDSAR |
| | | | LQMHWELWSKPVSELRANFTLQDRGAGPRVEMICQASSGSPPIT |
| | | | NSLIGKDGQVHLQQRPCHRQPANFSFLPSQTSDWF |
| NO: 11 | YM1672 | ENSP00000315731 | AQYSSDRCSWKGSGLTHEAHRKEVEQVYLRCAAGAVEWMYPTG |
| | | | ALIVNLRPNTFSPARHLTVCIRSFTDSSGANIYLEKTGELRLLVPDG |
| | | | DGRPGRVQCFGLEQGGLFVEATPQQDIGRRTTGFQYELVRRHR |
| | | | ASDLHELSAPCRPCSDTEVLLAVCTSDFAVRGSIQQVTHEPERQD |
| | | | SAIHLRVSRLYRQKSRVFEPVPEGDGHWQGRVRTLLECGVRPGH |
| | | | GDFLFTGHMHFGEAR |
| NO: 12 | YM1708 | ENSP00000392824 | MFGVLEGAQANSENWIAPSGPWALGLWSSLYFLLFSTLEGRGGR |
| | | | VLSQSCSMAVAAASWISRENARSVKRSYMQSSPQRPKEPRNQR |
| | | | TSHTTPVC |
| NO: 13 | YM1801 | ENSP00000367965 | SGFTALHWAAKSGDGEMALQLVEVARRSGAPVDVNARSHGGYT |
| | | | PLHLAALHGHEDAAVLLVVRLGAQVHVRDHSGRRAYQYLRPGSS |
| | | | YALRRLLGDPGLRGTTEPDATGGGSGSLAARRPVQVAATILSSTT |
| | | | SAFLGVLADDLMLQDLARGLKKSSSFSKFLSASPMAPRKKTKIRG |
| | | | GLPAFSEISRRPTPGPLAGLVPSFPPTT |
| NO: 14 | YM1882 | ENSP00000395006 | SLIVFMEQVHRGIKGLVRDSHGKGIPNAIISVEGINHDIRTANDGDY |
| | | | WRLLNPGEYVVTAKAEGFTASTKNCMVGYDMGATRCDFTLSKTN |
| | | | MARIREIMEKFGKQPVSLPARRLKLRGQKRRQRG |
| NO: 15 | YM1980 | ENSP00000362968 | GSLSPTKYNLLELKESCIRNQDCETGCCQRAPDNCESHCAEKGS |
| | | | EGSLCQTQVFFGQYRACPCLRNLTCIYSKNEKWLSIAYGRCQKIG |
| | | | RQKLAKKMFF |
| NO: 16 | YM1989 | ENSP00000415977 | SQLELIDLSSNPFHCDCQLLPLHRWLTGLNLRVGATCATPPNARG |
| | | | QRVKAAAAVFEDCPGWAARKAKRTPASRPSARRTPIKGRQCGA |
| | | | DKVGHGAGGV |
| NO: 17 | YM2046 | ENSP00000360191 | GEAGGSCLRWEPHCQQPLPDRVPSTAILPPRLNGPWISTGCEVR |
| | | | PGPEFLTRAYTFYPSRLFRAHQFYYEDPFCGEPAHSLLVKGKVRL |
| | | | RRASWVTRGATEADYHLHKVGIV |
| NO: 18 | YM2213 | ENSP00000414589 | ATKLVTCPAPRQFAVGAFTAAGRAWLFAPSLGASFSKLRSQQRS |
| | | | RDFRGRLFLRAERRAGGFTS |
| NO: 19 | YM2273 | ENSP00000374868 | SSNLEGRTKSVIRQTGSSAEITCDLAEGSTGYIHWYLHQEGKAPQ |
| | | | RLLYYDSYTSSVVLESGISPGKYDTYGSTRKNLRMILRNLIENDSG |
| | | | VYYCATWDGHSDSDPPYTTLKTCLVAASPREEGMRWALLVLLAF |
| | | | LSPIPAPPTVFCARDHFLVEWDLSFERIFYSFSSGPFPSKAPERKA |
| | | | CSQKSSNLEGRMKSVTRPTGSSAEITCDLTVINAVYIHWYLQQEG |
| | | | KTPQHLLHYDV |
| NO: 20 | YM2279 | ENSP00000374896 | AGVTQTPKFHVLKTGQSMTLLCAQDMNHEYMYRYRQDPGKGLR |
| | | | LIYYSVAAALTDKGEVPNGYNVSRSNTEDFPLKLESAAPSQTSVY |
| | | | FCASSYSTALQGCLLSAHKGKGRCCPPPPPKTQGCPVQRSLHQE |
| | | | PWNPEWPQVARTV |
| NO: 21 | YM2315 | ENSP00000374919 | AKVTQTPGHLVKGKGQKTKMDCTPEKGHTFVYWYQQNQNKEFM |
| | | | LLISFQNEQVLQETEMHKKRFSSQCPKNAPCSLAILSSEPGDTALY |
| | | | LCASSQSTALKCQFLLAHKLVTDPAQEAGDVLGWKGVTENNWSQ |
| | | | LKPQCNLTQG |
| NO: 22 | YM2671 | ENSP00000327628 | RDKMRMQRIKVCEKRPSIDLCIHHCSYFQKCETNKICCSAFCGNIC |
| | | | MSIL |
| NO: 23 | YM2741 | ENSP00000363414 | PERWFPGSCHVFGQGHQLFHIFLVLCTLAQLEAVALDYEARRPIY |
| | | | EPLHTHWPHN |
| NO: 24 | YM2779 | ENSP00000395093 | QLLMYQQHTSHYDLERKGGYLMLSFIDFCPFSVMRLRSLPSPQR |
| | | | YTRQERYRARPPRVLERSGFHNENSLAIYQGLVYYLLWLHSVYDK |
| | | | PYADPVHDPTWRWWANNKQDQDYYFFLASNWRSAGGVSIEMD |
| | | | SYEKIYNLESAYELPERIFLDKGTEYSFAIFLSAQGHSFRTQSELGT |
| | | | AFQLHSQVDVGVVLADPGCIEASVKQEVLINRNSVLFSITLKDKKL |
| | | | CYDQGISGHHLME |
| NO: 25 | YM2814 | ENSP00000258969 | VVEELKLSHNPLKSIPDNAFQSFGRYLETLWLDNTNLEKFSDGAFL |
| | | | GVTTLKHVHLENNRLNQLPSNFPFDSLETLALTNNPWKCTCQLRG |
| | | | LRRWLEAKASRPDATCASPAKFKGQHIRDTDAFRSCKFPTKRSK |
| | | | KAGRH |

In order to confirm the above results, a different set of proteins spotted in the microarray was used. This microarray now included all proteins identified as more reactive in the training set including the 24 proteins as indicated above (Table 4).

TABLE 4
Proteins printed on the focused microarray.

| | | | Protein |
|---|---|---|---|
| # | Accession Protein | Description | Array_Id |
| 1 | ENSP00000292144 | CD3g molecule, gamma (CD3-TCR complex) | YM0066 |
| 2 | ENSP00000418364 | CD80 molecule | YM0071 |
| 3 | ENSP00000377248 | CD86 molecule | YM0072 |
| 4 | ENSP00000228434 | CD69 molecule | YM0073 |
| 5 | ENSP00000216223 | interleukin 2 receptor, beta | YM0076 |
| 6 | ENSP00000379111 | interleukin 4 receptor | YM0078 |
| 7 | ENSP00000345501 | integrin, beta 7 | YM0079 |
| 8 | ENSP00000390133 | transferrin receptor (p90, CD71) | YM0083 |
| 9 | ENSP00000262817 | Transmembrane protein 59-like Precursor | YM0099 |
| 10 | ENSP00000361001 | EF-hand domain-containing protein KIAA0494 | YM0100 |
| 11 | ENSP00000363041 | CDGSH iron sulfur domain-containing protein 1 | YM0107 |
| 12 | ENSP00000296955 | Discoidin, CUB and LCCL domain-containing protein | YM0117 |
| | | 1 Precursor | |
| 13 | ENSP00000386923 | Lysozyme g-like protein 1 Precursor | YM0120 |
| 14 | ENSP00000302046 | Ribonuclease K6 Precursor | YM0125 |
| 15 | ENSP00000317385 | GLIPR1-like protein 2 | YM0724 |
| 16 | ENSP00000334044 | Ubiquitin-like protein 4B | YM0728 |
| 17 | ENSP00000350556 | Uncharacterized protein C19orf47 | YM0736 |
| 18 | ENSP00000312599 | Transmembrane protein 70, mitochondrial Precursor | YM0757 |
| 19 | ENSP00000334308 | Nesprin-3 | YM0838 |
| 20 | ENSP00000258436 | Major facilitator superfamily domain-containing | YM0872 |
| | | protein 9 | |
| 21 | ENSP00000357480 | Nogo-B receptor Precursor | YM0887 |
| 22 | ENSP00000412308 | Uncharacterized protein C18orf19 | YM0908 |
| 23 | ENSP00000364502 | Protein FAM70B | YM0972 |
| 24 | ENSP00000294258 | Zinc finger protein-like 1 | YM1020 |
| 25 | ENSP00000262262 | CD33 molecule | YM1046 |
| 26 | ENSP00000364621 | Sushi domain-containing protein 3 | YM1073 |
| 27 | ENSP00000266943 | Solute carrier family 46 member 3 Precursor | YM1077 |
| 28 | ENSP00000292301 | chemokine (C-C motif) receptor 2 | YM1099 |
| 29 | ENSP00000317300 | Lysophosphatidylcholine acyltransferase 4 | YM1129 |
| 30 | ENSP00000326267 | EF-hand domain-containing protein C14orf143 | YM1131 |
| 31 | ENSP00000356048 | Synaptotagmin-like protein 3 | YM1134 |
| 32 | ENSP00000342493 | Regulator of microtubule dynamics protein 3 | YM1183 |
| 33 | ENSP00000352601 | Low-density lipoprotein receptor-related protein | YM1204 |
| | | 10 Precursor | |
| 34 | ENSP00000347152 | Attractin-like protein 1 Precursor | YM1208 |
| 35 | ENSP00000416040 | UPF0606 protein KIAA1549 | YM1214 |
| 36 | ENSP00000248668 | Leucine-rich repeat and fibronectin type III | YM1217 |
| | | domain- containing protein 1 Precursor | |
| 37 | ENSP00000357631 | Transcription elongation regulator 1-like protein | YM1340 |
| 38 | ENSP00000311657 | GRAM domain-containing protein 2 | YM1342 |
| 39 | ENSP00000360822 | Potassium channel subfamily T member 1 | YM1343 |
| 40 | ENSP00000418985 | Solute carrier family 22 member 23 | YM1351 |
| 41 | ENSP00000311307 | Uncharacterized protein C4orf26 Precursor | YM1401 |
| 42 | ENSP00000404259 | EGF-like domain-containing protein 8 Precursor | YM1414 |
| 43 | ENSP00000384553 | Zinc/RING finger protein 3 Precursor | YM1423 |
| 44 | ENSP00000343084 | Putative uncharacterized protein | YM1451 |
| 45 | ENSP00000374125 | Sialic acid-binding Ig-like lectin 15 Precursor | YM1477 |
| 46 | ENSP00000375259 | Putative uncharacterized protein DKFZp667F0711 | YM1503 |
| | | Fragment | |
| 47 | ENSP00000263569 | Platelet receptor Gi24 Precursor | YM1526 |
| 48 | ENSP00000247618 | Kinesin light chain 1 | YM1529 |
| 49 | ENSP00000368502 | Secretoglobin-like protein Precursor | YM1534 |
| 50 | ENSP00000288466 | zinc finger protein 618 | YM1535 |
| 51 | ENSP00000359571 | Uncharacterized protein CXorf66 Precursor | YM1536 |
| 52 | ENSP00000315554 | Spermatid maturation protein 1 | YM1537 |
| 53 | ENSP00000415978 | Protein FAM74A1/A2 | YM1542 |
| 54 | ENSP00000325525 | similar to Killer cell immunoglobulin-like receptor | YM1546 |
| | | 3DL2 precursor (MHC class I NK cell receptor) | |
| | | (Natural killer associated transcript 4) (NKAT-4) | |
| | | (p70 natural killer cell receptor clone CL-5) | |
| 55 | ENSP00000406884 | HCG2023280cDNA FLJ30064 fis, clone | YM1587 |
| | | ADRGL2000323; | |
| 56 | NP_001034884 | Putative uncharacterized protein | YM1593 |
| 57 | ENSP00000374867 | hypothetical protein LOC648852 | YM1594 |
| 58 | ENSP00000397690 | Putative uncharacterized protein UNQ6190/ | YM1599 |
| | | PRO20217 Precursor | |
| 59 | XP_934154 | Putative uncharacterized protein | YM1602 |
| 60 | ENSP00000343493 | Uncharacterized protein C17orf99 Precursor | YM1651 |
| 61 | ENSP00000413076 | Uncharacterized protein C17orf99 Precursor | YM1652 |
| 62 | ENSP00000368396 | UPF0631 protein HSD24 | YM1668 |
| 63 | ENSP00000328061 | Uncharacterized protein C17orf74 | YM1671 |
| 64 | ENSP00000315731 | Meteorin-like protein Precursor | YM1672 |
| 65 | ENSP00000331466 | Transmembrane protein 95 Precursor | YM1674 |
| 66 | ENSP00000331466 | Transmembrane protein 95 Precursor | YM1675 |
| 67 | ENSP00000304670 | RING finger and transmembrane domain-containing | YM1684 |
| | | protein 1 | |
| 68 | ENSP00000303437 | t-SNARE domain-containing protein 1 | YM1693 |
| 69 | ENSP00000392824 | LP5624 | YM1708 |
| 70 | ENSP00000346747 | Putative uncharacterized protein | YM1720 |
| 71 | ENSP00000262424 | Cysteine-rich secretory protein LCCL domain- | YM1770 |
| | | containing 2 Precursor | |
| 72 | ENSP00000360577 | Enoyl-CoA hydratase domain-containing protein | YM1778 |
| | | 2, mitochondrial Precursor | |
| 73 | ENSP00000367965 | Ankyrin repeat domain-containing protein 43 | YM1801 |
| | | Precursor | |
| 74 | ENSP00000354240 | RPE-spondin Precursor | YM1810 |
| 75 | OTTHUMP00000028326 | Putative uncharacterized protein | YM1824 |
| 76 | ENSP00000395006 | Carboxypeptidase-like protein X2 Precursor | YM1882 |
| 77 | ENSP00000298966 | UPF0443 protein C11orf75 | YM1909 |
| 78 | ENSP00000350961 | Transmembrane protein C9orf123 | YM1914 |
| 79 | ENSP00000378605 | DnaJ homolog subfamily C member 30 | YM1925 |
| 80 | OTTHUMP00000028097 | Putative uncharacterized protein | YM1957 |
| 81 | ENSP00000368662 | Uroplakin-3-like protein Precursor | YM1960 |
| 82 | ENSP00000405827 | Putative uncharacterized protein | YM1971 |
| 83 | ENSP00000362968 | Colipase-like protein C6orf127 Precursor | YM1980 |
| 84 | ENSP00000409535 | C-type lectin domain family 18 member B | YM1985 |
| | | Precursor | |
| 85 | ENSP00000415977 | Chondroadherin-like protein Precursor | YM1989 |
| 86 | ENSP00000412448 | | YM2013 |
| 87 | ENSP00000414449 | | YM2036 |
| 88 | ENSP00000360191 | Protein APCDD1-like Precursor | YM2046 |
| 89 | ENSP00000321517 | Putative uncharacterized protein | YM2054 |
| 90 | ENSP00000339578 | Seven transmembrane helix receptor | YM2071 |
| 91 | ENSP00000397696 | HSAL5836 | YM2094 |
| 92 | ENSP00000366415 | Putative uncharacterized protein | YM2100 |
| 93 | ENSP00000345107 | Putative uncharacterized protein | YM2110 |
| 94 | ENSP00000344029 | Putative uncharacterized protein UNQ9165/ | YM2111 |
| | | PRO28630 Precursor | |
| 95 | ENSP00000409458 | Putative uncharacterized protein | YM2136 |
| 96 | ENSP00000398103 | Putative uncharacterized protein UNQ6493/ | YM2140 |
| | | PRO21345 | |
| 97 | ENSP00000372305 | Putative uncharacterized protein | YM2144 |
| 98 | ENSP00000411889 | Putative uncharacterized protein UNQ6490/ | YM2151 |
| | | PRO21339 Precursor | |
| 99 | ENSP00000349132 | Protein FAM75A3 | YM2168 |
| 100 | ENSP00000406884 | HCG2023280cDNA FLJ30064 fis, clone | YM2202 |
| | | ADRGL2000323; | |
| 101 | ENSP00000414589 | VGSA5840 | YM2213 |
| 102 | ENSP00000389279 | AHPA9419 | YM2214 |
| 103 | ENSP00000358798 | Calcium homeostasis modulator protein 3 | YM2223 |
| 104 | ENSP00000374897 | | YM2237 |
| 105 | ENSP00000373765 | monooxygenase, DBH-like 2 pseudogene (MOXD2) | YM2250 |
| 106 | ENSP00000374869 | | YM2251 |
| 107 | ENSP00000304930 | Sclerostin domain-containing protein 1 | YM2255 |
| | | Precursor | |
| 108 | ENSP00000409076 | Uncharacterized protein KIAA1644 Precursor | YM2271 |
| 109 | ENSP00000374868 | Putative uncharacterized protein | YM2273 |
| | | ENSP00000374868 | |
| 110 | ENSP00000374880 | | YM2276 |
| 111 | ENSP00000374896 | Putative uncharacterized protein | YM2279 |
| | | ENSP00000374896 | |
| 112 | ENSP00000400516 | Hematopoietic cell signal transducer Precursor | YM2284 |
| 113 | ENSP00000382000 | CMT1A duplicated region transcript 15 protein- | YM2286 |
| | | like protein | |
| 114 | ENSP00000311857 | | YM2289 |
| 115 | ENSP00000331418 | | YM2297 |
| 116 | ENSP00000374919 | Putative uncharacterized protein | YM2315 |
| | | ENSP00000374919 | |
| 117 | OTTHUMP00000076641 | Putative uncharacterized protein | YM2322 |
| 118 | OTTHUMP00000076959 | Putative uncharacterized protein | YM2326 |
| 119 | ENSP00000222033 | Zinc/RING finger protein 4 Precursor | YM2335 |
| 120 | ENSP00000251473 | Lipid phosphate phosphatase-related protein | YM2355 |
| | | type 2 | |
| 121 | ENSP00000291934 | Transmembrane protein 190 Precursor | YM2388 |
| 122 | ENSP00000393015 | Serine protease 23 Precursor | YM2441 |
| 123 | ENSP00000414523 | Leucine-rich repeat transmembrane neuronal | YM2448 |
| | | protein 1 Precursor | |
| 124 | ENSP00000363345 | Thymic stromal cotransporter homolog | YM2453 |
| 125 | ENSP00000307164 | Prostate and testis expressed protein 1 | YM2511 |
| | | Precursor | |
| 126 | ENSP00000355243 | Protocadherin-7 Precursor | YM2570 |
| 127 | ENSP00000264661 | Potassium voltage-gated channel subfamily H | YM2576 |
| | | member 4 | |
| 128 | ENSP00000266646 | Inhibin beta E chain Precursor | YM2613 |
| 129 | ENSP00000344847 | A disintegrin and metalloproteinase with | YM2616 |
| | | thrombospondin motifs 12 Precursor | |
| 130 | ENSP00000298690 | Ribonuclease 7 Precursor | YM2626 |
| 131 | ENSP00000321345 | interleukin 23 receptor | YM2646 |
| 132 | ENSP00000361735 | WAP four-disulfide core domain protein 8 | YM2665 |
| | | Precursor | |
| 133 | ENSP00000327628 | Protein WFDC10B Precursor | YM2671 |
| 134 | ENSP00000259216 | Cryptic protein Precursor | YM2707 |
| 135 | ENSP00000349131 | R-spondin-3 Precursor | YM2726 |
| 136 | ENSP00000347451 | Leucine-rich repeat and immunoglobulin-like | YM2728 |
| | | domain- containing nogo receptor-interacting | |
| | | protein 1 Precursor | |
| 137 | ENSP00000363414 | Membrane progestin receptor alpha | YM2741 |
| 138 | ENSP00000385766 | Leucine-rich repeat-containing protein 32 | YM2767 |
| | | Precursor | |
| 139 | ENSP00000395093 | Uncharacterized protein C19orf15 Precursor | YM2779 |
| 140 | ENSP00000258969 | Chondroadherin Precursor | YM2814 |
| 141 | ENSP00000272134 | left-right determination factor 1 | YM2826 |
| 142 | ENSP00000215885 | Group 3 secretory phospholipase A2 Precursor | YM2831 |
| 143 | ENSP00000255039 | Hyaluronan and proteoglycan link protein 2 | YM2843 |
| | | Precursor | |
| 144 | ENSP00000380417 | Interleukin-25 Precursor | YM2854 |

In order to confirm the above results, the authors probed the focused protein microarray with a second serum sample set (Test Set as indicated in Table 1 Material and Methods) comprising other 15 sera of patients with AILD and 39 serum samples from healthy subjects. Following the same normalization and using criteria described above (see Materials and Methods), the authors confirmed that the 25 autoantigens were differentially recognized by patients compared to healthy donors with statistical significance. Interestingly, in unsupervised hierarchical clustering analysis these autoantigens allowed for good discrimination of the two populations of sera in both sample sets, as shown in FIG. 4a.

Having identified 25 autoantigens highly recognized by AILD patients, the authors asked whether non-autoimmune liver disease patients had an overlapped recognition pattern. They therefore tested the same microarray with sera from 110 patients with chronic HCV infection (Table 1). FIG. 4b shows the MFIs of the 25 autoantigens in each group of individuals (AILD, HD and HCV respectively), and highlights that, although some reactivity is observed in patients affected from HCV, 17 auto-antigens react preferentially and more strongly with sera from AILD patients, with statistical significance (p val <0.01).

### Example 4

### Validation of Selected Autoantigens with an Independent Sample Set Confirms that 6 Proteins are New Potential AILD Biomarkers

After having identified a total of 17 auto-antigens specific for autoimmune patients by protein microarray, the authors validated the results obtained by using the Dissociation-Enhanced Lanthanide Fluorescence ImmunoAssay method (DELFIA®) assay method (Materials and Methods).

By this assay the authors screened an independent sample set (Validation set as indicated in Table 1, Materials and Methods) comprising 100 AILD patients (50 AIH and 50 PBC), 50 healthy donors and 74 patients with chronic viral hepatitis (50 HCV and 24 HBV) measured by time-resolved fluorescence. All sera were tested at a dilution of 1:300 as described in Materials and Methods, and the antigen-specific IgG responses to each of the 17 selected autoantigens was measured by time-resolved fluorescence. Reproducibility of the results was confirmed using duplicate sample of selected sera.

All 17 antigens displayed higher mean fluorescence intensity compared to HD and chronic viral hepatitis (FIG. 5). Fluorescence intensity values higher than the mean plus three standard deviations of buffer signals among healthy donors were considered positive. As shown in Table 5, 6 of the 17 antigens displayed significantly higher recognition frequency by AILD patients compared to healthy donors, and were also significant when compared to viral hepatitis patients (FIG. 6), thus being considered as top candidates.

TABLE 5
Recognition frequencies of individual antigens identified
as candidate AILD biomarkers in the validation sample set.

| | | | Positive sera % | | |
|---|---|---|---|---|---|
| | | | HD | AILD | VH |
| Description | Prot. Id | Combo^{A} | (n = 50) | (n = 100) | (n = 74) |
| Asialoglyco- | AGPR | | 50 | 70 | 53 |
| protein receptor | | | | | |
| Cytochrome | Cyp450 | | 56 | 72 | 55 |
| P4502D6 | | | | | |
| Pyruvate | PDH | | 0 | 55 | 4 |
| dehydrogenase | | | | | |
| Interleukin-4 | YM0078 | x∘• | 2 | 74 | 14 |
| receptor domain | | | | | |
| Putative | YM1503 | x | 6 | 48 | 14 |
| uncharacterized | | | | | |
| protein | | | | | |
| Putative | YM1602 | x | 8 | 44 | 14 |
| uncharacterized | | | | | |
| protein | | | | | |
| Uncharacterized | YM1652 | x∘ | 4 | 47 | 15 |
| protein C17orf99 | | | | | |
| Meteorin-like | YM1672 | x∘• | 0 | 48 | 22 |
| protein Precursor | | | | | |
| Protein APCDD1- | YM2046 | x∘• | 2 | 63 | 28 |
| like Precursor | | | | | |

HD: Healthy donors; AILD: autoimmune liver disease patients, VH: Viral hepatitis patients.
^{A}Antigens used for combination assays comprising 6 (x), 4 (∘) and 3 (•) markers respectively.
SE % = percentage of positive AIH sera; SP % = percentage of negative HD or VH sera.

These top 6 antigens, showed high sensitivity (from 44 to 74% of positive AILD patients) and specificity (from 92 to 100% of negative HD). Interestingly, individual sensitivity was comparable to that obtained in our hands by 3 known AILD markers, CYP2D6 (Cytochrome Peroxidase 2D6), AGPR-1 (Asialo-Glycoprotein Receptor 1) and PDH (Pyruvate DeHydrogenase), (Jensen, D M, 1978, The New England journal of medicine; Van de Water J. et al., 1993, The Journal of clinical investigation), while individual specificity was far better for our candidates compared to the benchmarks.

The authors next assessed the discrimination power of combinations of the six autoantigens. We therefore tested combinations of three (YM0078, YM1672, YM2046), four (YM0078, YM1652, YM1672, YM2046) or six proteins (YM1672, YM0078, YM2046, YM1652, YM1503, YM1602). FIG. 7a shows that combinations allow clear discrimination between patients with AILD and healthy donors, with sensitivities of 77%±4 (86, 81 and 79%, for 3, 4 and 6 combos) and specificities of 91%±8 (96, 92 and 82% respectively). Moreover, FIG. 7b shows the ROC curves of logistic regression models obtained with the same combinations of new antigens compared to the combination of the three known control proteins, and illustrates that any of the new antigens combo is superior to the combo of the three known control proteins.

### REFERENCES

1. Czaja, A. J., and Manns, M. P. 2010. Gastroenterology 139:58-72 e54.
2. Makol, A., Watt, K. D., and Chowdhary, V. R. 2011. 2011:390916.
3. Bogdanos D P, Mieli-Vergani G, Vergani D. 2009. 29(3):241-53.
4. Selmi, C., Mackay, I. R., and Gershwin, M. E. 2011. 89:70-80.
5. Muratori L, Muratori P, Granito A, Pappas G, Cassani F, Lenzi M. 2010. Dig Liver Dis 42(11):757-64.
6. Muratori, L., Granito, A., Muratori, P., Pappas, G., and Bianchi, F. B. 2008. Clinics in liver disease 12:261-276; vii.
7. Bogdanos, D. P., Invernizzi, P., Mackay, I. R., and Vergani, D. 2008. World journal of gastroenterology: WJG 14:3374-3387.
8. Vergani, D., Alvarez, F., Bianchi, F. B., Cancado, E. L., Mackay, I. R., Manns, M. P., Nishioka, M., and Penner, E. 2004. Journal of hepatology 41:677-683.
9. Zachou, K., Rigopoulou, E., and Dalekos, G. N. 2004. Journal of autoimmune diseases 1:2.
10. Song, Q., Liu, G., Hu, S., Zhang, Y., Tao, Y., Han, Y., Zeng, H., Huang, W., Li, F., Chen, P., et al. 2010. 9:30-39.
11. Grifantini, R., Pagani, M., Pierleoni, A., Grandi, A., Parri, M., Campagnoli, S., Pileri, P., Cattaneo, D., Canidio, E., Pontillo, A., et al. 2011. Journal of proteomics 75:532-547.
12. Bombaci, M., Grifantini, R., Mora, M., Reguzzi, V., Petracca, R., Meoni, E., Balloni, S., Zingaretti, C., Falugi, F., Manetti, A. G., et al. 2009. PloS one 4:e6332.
13. Harris, J. W. and Stocker, H.1998. Handbook of Mathematics and Computational Science. New York: Springer-Verlag.
14. Saeed, A. I., Bhagabati, N. K., Braisted, J. C., Liang, W., Sharov, V., Howe, E. A., Li, J., Thiagarajan, M., White, J. A., and Quackenbush, J. 2006. Methods in enzymology 411:134-193.
15. Frulloni, L., Lunardi, C., Simone, R., Dolcino, M., Scattolini, C., Falconi, M., Benini, L., Vantini, I., Corrocher, R., and Puccetti, A. 2009. The New England journal of medicine 361:2135-2142.
16. Sing, T., Sander, O., Beerenwinkel, N., and Lengauer, T. 2005. ROCR: visualizing classifier performance in R. Bioinformatics 21:3940-3941.
17. Jensen, D. M., McFarlane, I. G., Portmann, B. S., Eddleston, A. L., and Williams, R. 1978. The New England journal of medicine 299:1-7.
18. Van de Water, J., Turchany, J., Leung, P. S., Lake, J., Munoz, S., Surh, C. D., Coppel, R., Ansari, A., Nakanuma, Y., and Gershwin, M. E. 1993. The Journal of clinical investigation 91:2653-2664.

1. An in vitro method of diagnosis or prognosis or evaluation of risk to develop a liver autoimmune disorder belonging to the group of AIH and PBC in a subject, comprising the steps of:
a) contacting a biological sample from the subject with a protein selected from the group consisting of: a protein having the amino acid sequence SEQ ID No. 1, an allelic variant, an orthologous, at least one immunological fragment and functional equivalents thereof, under conditions appropriate for binding of autoantibodies, if present in the biological sample, to said protein, and
b) detecting the presence of bound autoantibodies.

2. The method according to claim 1 wherein step a) is performed by contacting said biological sample with the protein as in claim 1 and at least one further protein selected from the group consisting of 16 proteins having amino acid sequence SEQ ID Nos. 2, 3, 5, 6, 7, 8, 9, 10, 11, 12, 14, 15, 16, 17, 22, 25, allelic variants, orthologous, immunological fragments and functional equivalents thereof.

3. The method according to claim 2 wherein step a) is performed by contacting said biological sample with three proteins having amino acid sequence SEQ ID Nos. 1, 11, 17, allelic variants, orthologous, immunological fragments or functional equivalents thereof.

4. The method according to claim 3 wherein step a) is performed by contacting said biological sample with four proteins having amino acid sequence SEQ ID Nos. 1, 10, 11, 17, allelic variants, orthologous, immunological fragments or functional equivalents thereof.

5. The method according to claim 4 wherein step a) is performed by contacting said biological sample with six proteins having amino acid sequence SEQ ID Nos. 1, 6, 8, 10, 11, 17, allelic variants, orthologous, immunological fragments or functional equivalents thereof.

6. The method according to claim 1 wherein the biological sample is selected from the group consisting of blood, serum, plasma, urine, saliva, mucus, and fractions thereof.

7. The method of claim 1 wherein the biological sample is from an adult or from an adolescent.

8. The method according to claim 1 wherein the detection of said bound autoantibodies is performed by means of binding to specific ligands.

9. The method according to claim 8 wherein the ligands are conjugated with detecting means.

10. A method of monitoring an autoimmune liver disorder after treatment with surgery and/or therapy in a subject with said autoimmune liver disorder, comprising the step of following the modulation of proteins as claimed in claim 1.

11. The method of claim 1, wherein said proteins or functional equivalents thereof are displayed on one or more protein microarrays.

12. (canceled)

13. A solid support for an immunodiagnostic assay comprising a protein of claim 1 or immunological fragments or functional equivalents thereof.

14. An immunodiagnostic kit comprising the solid support of claim 13 and detecting means.
